(19) United States
(12) Patent Application Publication
(10) Pub. No.: US 2005/0119531 A1
(43) Pub. Date: Jun. 2, 2005

(54) Method of performing shoulder surgery

(75) Inventor: Todd William Sharratt, Birchwood, MN (US)

Correspondence Address: Z. Peter Sawicki;Westman, Champlin & Kelly, 900 Second Avenue South, Suite 1600, Minneapolis, MN, 55402-3319, US

(73) Assignee: Minnesota Scientific, Inc., St. Paul, MN (US)

(21) Appl. No.: 11/029,263

(22) Filed: Jan. 5, 2005

Related U.S. Application Data
(63) Continuation-in-part of application No. 10/728,202, filed on Dec. 4, 2003.
Application No. 10/728,202 is a continuation of application No. 10/077,693, filed on Feb. 15, 2002, now Pat. No. 6,659,944.
Application No. 10/077,693 is a continuation of application No. 09/990,420, filed on Nov. 21, 2001, now Pat. No. 6,368,271.
Application No. 09/990,420 is a continuation-in-part of application No. 09/654,400, filed on Sep. 1, 2000, now Pat. No. 6,315,718.
(63) Continuation-in-part of application No. 10/892,816, filed on Jul. 16, 2004.
Application No. 10/892,816 is a continuation-in-part of application No. 10/623,179, filed on Jul. 18, 2003.
(60) Provisional application No. 60/396,850, filed on Jul. 18, 2002.

(51) Int. Cl. A61B 1/32

(52) U.S. Cl. 600/227; 623/19.11; 623/19.13; 606/86

(57) ABSTRACT

A method of performing a shoulder joint surgery involving a scapula having a glenoid cavity, a humerus and a humeral ball on a surgical table includes mounting a retractor support to the surgical table and positioning the retractor support about the shoulder joint. The skin and flesh layers proximate the shoulder joint are incised and the skin and flesh layers are manually retracted with a retractor. The retractor is secured in a selected position by attaching the retractor to the retractor support.

### CROSS REFERENCE TO RELATED APPLICATIONS

This application is a continuation-in-part of application Ser. No. 10/728,202 filed on Dec. 4, 2003, which is hereby incorporated by reference in its entirety, which is a continuation of application Ser. No. 10/077,693, filed Feb. 15, 2002, which is incorporated by reference in its entirety, and resulting in U.S. Pat. No. 6,659,944, which is a continuation of application Ser. No. 09/990,420 filed on Nov. 21, 2001, which is incorporated by reference in its entirety, and resulting in U.S. Pat. No. 6,368,271.

This application is also a continuation-in-part of application Ser. No. 10/892,816 filed on Jul. 16, 2004, which is hereby incorporated by reference in its entirety, which is a continuation-in-part of application Ser. No. 10/623,179; filed Jul. 18, 2003, which is hereby incorporated by reference in its entirety, which claims priority of U.S. Provisional Application No. 60/396,850, filed Jul. 18, 2002, the content of which is hereby incorporated by reference in its entirety.

### BACKGROUND OF THE INVENTION

The present invention relates to a method of surgical retraction. In particular, the present invention relates to a method of utilizing a table mounted retracting device during a surgical procedure on a shoulder of a patient.

Total shoulder replacement (arthroplasty) operations have been performed for many decades to repair shoulder components. These components include the glenoid cavity (socket portion of the shoulder) and the humeral head (ball portion of the shoulder). The shoulder is typically replaced due to deterioration or wear of the glenoid cavity and/or the humeral ball. Common causes of deterioration of the shoulder joint from smooth surfaces where the joint is properly articulating to rough surfaces where articulation of the joint is painful include osteoarthritis, Rheumatoid arthritis as well as trauma which injures the shoulder joint. The deterioration causes either the humeral ball, the glenoid cavity or both the humeral ball and the glenoid cavity become rough which results in pain when the shoulder joint is articulated.

Shoulder joint replacement procedures are successful in alleviating this type of shoulder pain. In a total shoulder replacement procedure, a cup shaped insert, typically manufactured of high density polyethylene, is inserted into the worn glenoid cavity and a metal ball is cemented to the humerus as a replacement to the humeral ball. In a partial shoulder replacement surgery, the worn component of the shoulder is replaced where either the humeral ball is replaced or an insert is positioned and retained in the glenoid cavity.

Typically, at least two physically strong people are needed to perform a total shoulder replacement. One person is needed to retract and retain the humerus from the glenoid cavity to gain access to both the glenoid cavity and the humerus. The other person is needed to prepare the glenoid cavity to accept the insert and also prepare the humerus to accept a metal ball.

After the replacement components are inserted, the shoulder is reducted (the humeral component is inserted into the glenoid cavity component) to check the angle and fit of the humeral ball into the glenoid cavity insert. If the shoulder components do not properly articulate, the shoulder joint is again dislocated, the components readjusted, and the humeral ball is repositioned in the glenoid cavity. When the stability and placement of the trial inserts are acceptable, the shoulder is dislocated and the humeral implant is secured to the humerus. The humeral ball is again positioned within the glenoid cavity and the stability of the arthroplasty is confirmed.

Due to the multiple dislocations and insertions of the humeral ball into the glenoid cavity, as well as retracting the humerus, the surgical procedure can become quite physically taxing on the surgeon or surgeons. The surgical procedure requires lifting and moving the patient's arm into multiple positions. At times, the surgeon may need to hold the arm in a selected position for an extended period of time. Depending on the size of the patient, the strenuous activity can lead to fatigue and contribute to surgical error.

Additionally, the repeated movement of the arm can cause nerve damage if it is not done precisely and with minimal adjustment. When the surgeon moves the humerus by hand it is common to have continual adjustment occur. Often, the surgeon holding the arm relaxes or becomes fatigued and allows the arm to move, requiring that the arm be readjusted. The movement can cause the arm to pinch or rub nerves or muscle tissue, possibly causing damage.

### BRIEF SUMMARY OF THE INVENTION

The present invention includes a method of performing a shoulder joint surgery involving a scapula having a glenoid cavity, a humerus and a humeral ball on a surgical table. The method includes mounting a retractor support to the surgical table and positioning the retractor support about the shoulder joint. The skin and flesh layers proximate the shoulder joint are incised and the skin and flesh layers are manually retracted with a retractor. The retractor is secured in a selected retracting position by attaching the retractor to the retractor support.

### BRIEF DESCRIPTION OF THE DRAWINGS

FIG. 1 is a perspective view of a table mounted support structure for a shoulder surgery.

FIG. 2 is a perspective view of an alternative embodiment of a table mounted support structure for a shoulder surgery.

FIG. 3 is a perspective view of the table mounted support structure exposing a shoulder joint for a complete shoulder reconstruction.

FIG. 4 is a perspective view of a reconstructed shoulder joint with a partial sectional view of a glenoid cavity within a scapula.

### DETAILED DESCRIPTION

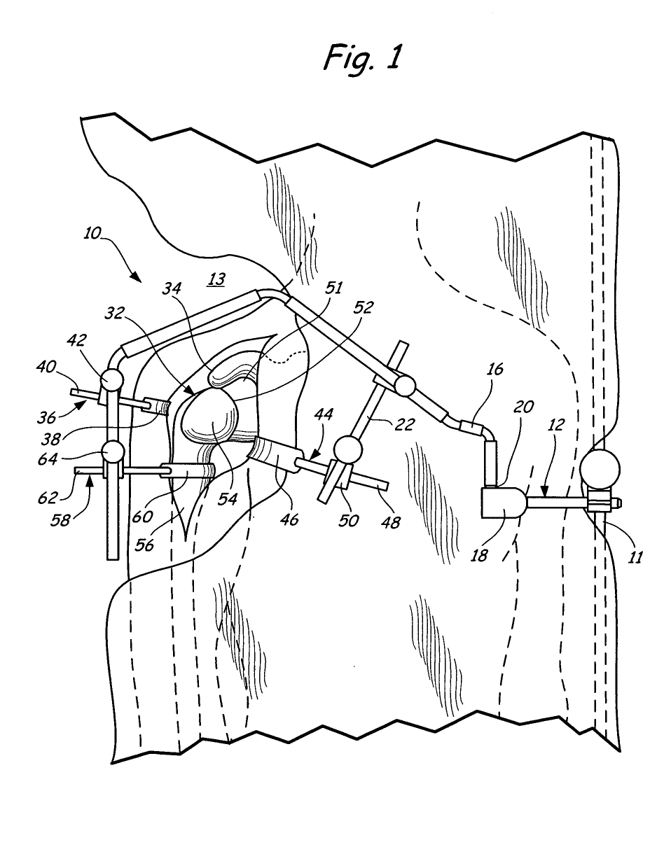

The present invention relates to a method of performing surgical procedures on a shoulder joint. An apparatus used in the surgical procedures of the present invention is generally indicated at 10 in FIG. 1.

The apparatus 10 includes a retractor support apparatus 12 that is rigidly mounted to a rail 11 of a surgical table 13 in a manner that is well known in the art and is described in U.S. Pat. Nos. 4,617,916, 4,718,151, 4,949,707, 5,400,772, 5,741,210, 6,042,541, 6,264,396 and 6,315,718 all of which are herein incorporated by reference. From the mount to the surgical table 13, the retractor support apparatus 12 includes at least one support arm that extends over the surgical table 13. The support arm 16 is removably attached to a clamping mechanism 18 having at least one pivot ball 20 rotatably movable within a clamping bore (not shown). The pivot ball 20 includes a cavity (not shown) into which an end of the support arm 16 is inserted. The support arm 16 is adjustable into an infinite number of selected positions by rotating the pivot ball 20 within the clamping bore (not shown).

The support arm 16 preferably has a configuration generally in the shape of the letter “J”. The generally “J” shaped support arm 16 provides a support structure about a shoulder area 30 that supports retractors that retract skin, muscle, blood vessels and nerves to expose the shoulder joint, as well as retract bone when the shoulder joint is separated. A rod 22 is preferably clamped to the generally “J” shaped support arm and extends over the patient to provide an additional support structure on an opposite side of a shoulder joint 32 from the generally “J” shaped support arm 16 while maintaining access thereto.

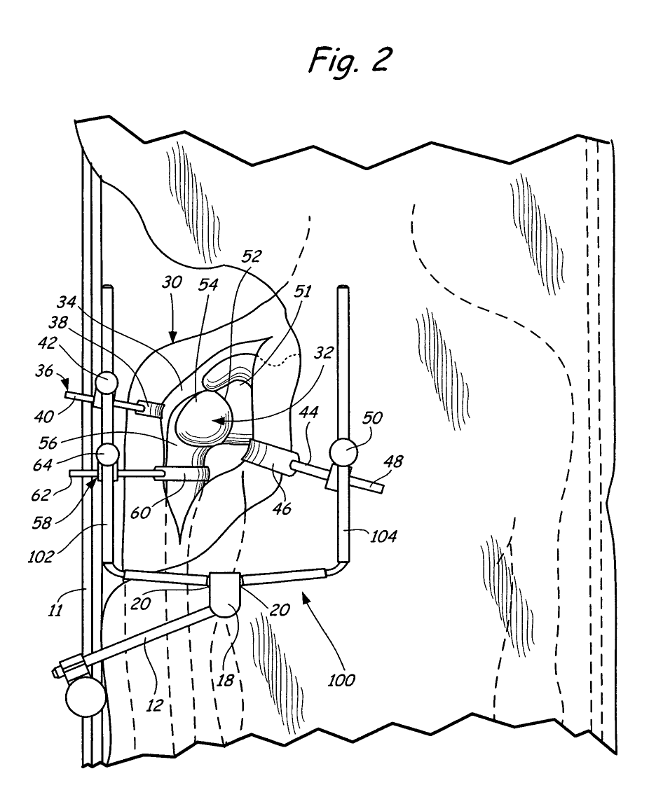

Alternatively, referring to FIG. 2, the apparatus 100 may include support arms 102 and 104 that are generally mirror images of each other that extend general horizontally over the surgical table 13 and are positioned on opposite sides the shoulder joint 30. The support arms 102 and 104, as well as the generally J-shaped support arm 16, are independently adjustable into an infinite number of selected positions through use of the clamping mechanism 20 which is described in U.S. Pat. Nos. 5,899,627 and 6,264,396, which are herein incorporated by reference. The clamp 20 secures the adjustable support arms 102 and 104 or the generally J-shaped support arm in selected angular positions with respect to the shoulder joint 30.

The surgical procedures on the shoulder joint 32 can be performed with any table mounted support structure and is not limited by the configurations illustrated and described herein. However, the table mounted support structure to must provide support for mounting retractors to retract skin, muscle, blood vessels, tendons and bone while providing access to the shoulder joint 32.

Referring to FIG. 1, with the retractor support apparatus 12 positioned in a selected position about the shoulder joint, an incision 34 is made through the skin on the front of the shoulder joint 32, otherwise known as an anterior approach to the shoulder joint 32. Typically, the incision is about three inches long but may vary depending upon the surgical procedure being performed.

After making the incision 34 through the skin, the surgeon divides the tissue, muscle, blood vessels and nerves to expose the shoulder joint while causing minimal trauma. To expose the shoulder joint 32, a retractor blade 38 of a lateral retractor 36 is positioned within the incision 34. The tissue, muscle, blood vessels and nerves are laterally retracted from the shoulder joint 32 with manual force applied to the lateral retractor 36. With the lateral retractor in the selected position, a handle 40 of the lateral retractor 36 is positioned within a clamping socket (not shown) of a clamp 42 positioned on the J shaped support arm 16 and is secured in the selected position by positioning the clamp 42 in the clamping position. Preferably, the clamp socket (not shown) allows the retractor handle to be manually forced therein without having to position an end of the handle through the clamp, although other clamps are within the scope of the present invention. By socket is meant an opening or a cavity into which an inserted part, such as a retractor support apparatus, is designed to fit and wherein the retractor support apparatus can be inserted into the socket from an infinite number of directions in a 180° range starting from a substantially parallel position to a back surface of the socket to a position substantially perpendicular to the back surface and continuing to position again substantially parallel to the back surface of the socket. By lateral is meant a position or direction generally away from the body.

A retractor blade 46 of a medial retractor 44 is positioned within the incision 32 generally opposite the lateral retractor 36. Manual force is applied to the medial retractor 44 to retract tissue, muscle, blood vessels and nerves in a medial direction from the shoulder joint 32. With the medial retractor 44 in a selected position, a handle 48 of the medial retractor 44 is positioned within a clamping socket (not shown) of a clamp 50 positioned on the rod 22 and secured in the selected position by positioning the clamp in the clamping position. By medial is meant a position or direction toward the body of the patient. Although a clamp with a socket is preferred, clamps with differently configured clamping surfaces are also within the scope of the present invention including a clamping bore.

Although two retractors 36, 44 are a preferred number of retractors for exposing the surgical site in the shoulder, more than two retractors supported by the table mounted retractor support apparatus 12 can be utilized to expose the surgical site in the shoulder area 30 while performing the surgical procedure of the present invention. Mechanical mechanisms on retractors may be used to adjust the vertical position of the retractor blade and the lateral or medial position of the retractor blade within the surgical site. Mechanical mechanism used to adjust the position of the retractor blade include, but are not limited to, articulated joints, rack and pinion systems, racheting mechanisms, wedges, ramps, camming mechanisms and threadable engagements are within the scope of the present invention.

With the shoulder joint 32 exposed by the retraction of the skin, tissue, muscle, blood vessels and nerves, the surgical procedure on the shoulder joint 32 can be performed. A non-exhaustive list of surgical procedures that can be performed on the shoulder include repair of a muscle tear, repair of a torn or ruptured tendon or ligament such as a torn rotator cuff, as well as a shoulder replacement surgery. The shoulder replacement surgery includes a complete shoulder replacement and a partial shoulder replacement surgery.

In a shoulder replacement surgery, the skin, muscle, blood vessels and nerves are preferably retracted without having to be severed with a scalpel to expose a shoulder joint 32 with at least the lateral and medial retractors 36, 44, respectively. Performing the shoulder surgery while minimizing the damage the muscle, blood vessels and nerves minimizes the post-operative pain felt by the patient as well as reducing the time required to rehabilitate the shoulder joint. However, it is within the scope of the present invention to perform a surgical procedure that requires incising muscles, blood vessels and nerves around the shoulder joint.

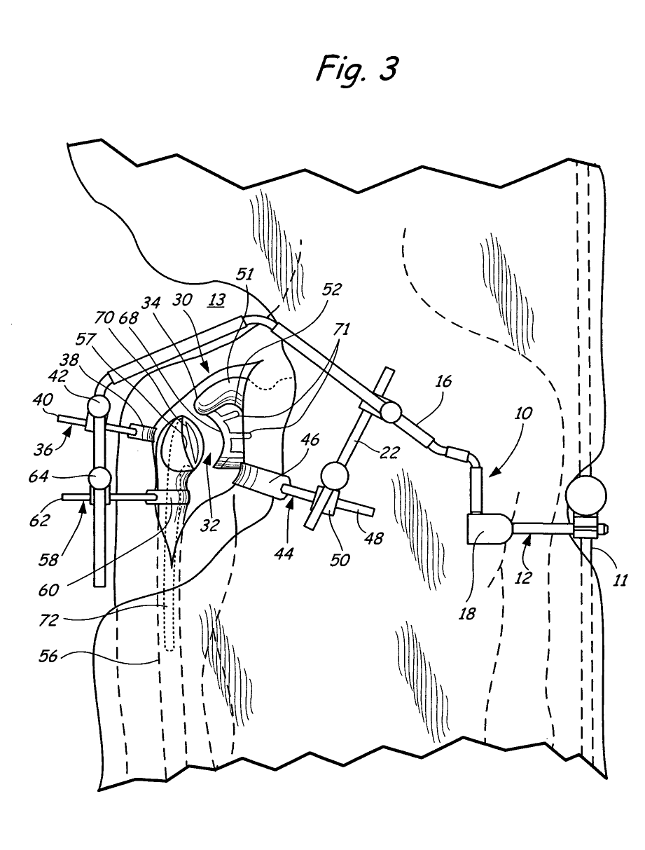

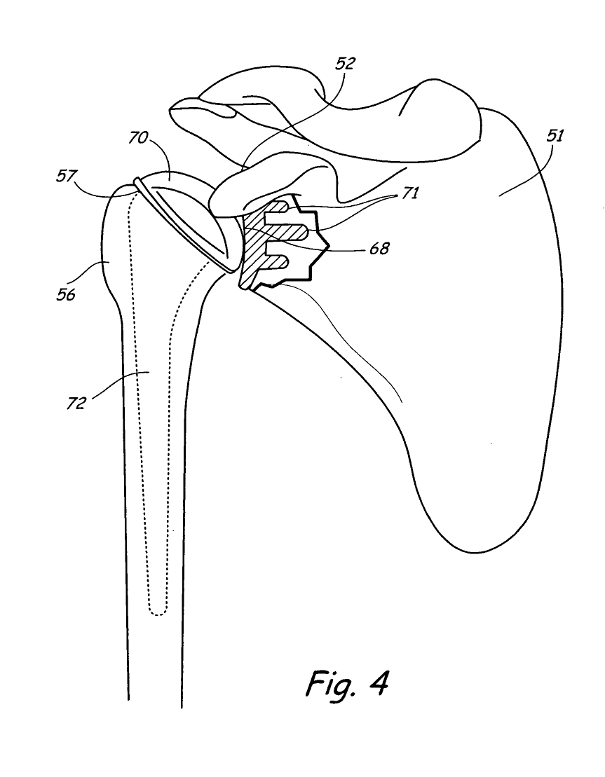

Referring to FIGS. 1, 3 and 4, with the shoulder joint 32 exposed, a scapula 51 having a glenoid cavity 52, a humeral ball 54 and an upper portion of a humerus 56 are viewable through the retracted incision 34. In preparing the shoulder joint 32 for the shoulder replacement surgery, the surgeon has at least two options in gaining access to the surfaces that accept inserts. First, the humeral ball 54 may be separated from the humerus 56, typically with a bone saw, while the humeral ball 54 remains within the glenoid cavity 52. The humerus 56 is retracted laterally away from the shoulder joint 32 to gain access to a freshly cut surface 57 on the humerus 56 onto which a humeral replacement insert 70 is affixed.

The humerus 56 is retracted with a retractor 58 having a blade 60 described in detail in U.S. Pat. Nos. 6,368,271 and 6,659,944, previously incorporated by reference, which is similar to a Fakuda blade, which is known in the art. The blade 60 includes a generally flat portion and an arcuate end that is positioned about the upper end of the humerus 56. The blade 60 also includes an aperture within both the generally flat portion and the arcuate end that aids in gripping the humerus and prevents the humerus from slipping along the blade. However, the aperture, while providing additional gripping capability to the retractor blade, is not necessary to retract the humerus from the glenoid cavity.

The humerus 56 can be retracted with manual force placed upon the retractor 58. With the humerus 56 manually retracted into a selected position, the retractor 58 is clamped to the retractor support apparatus 12 by positioning a retractor handle 62 into a clamping socket (not shown) of a clamp 64 and positioning the clamp 64 into the clamping position. Alternatively, the humerus 56 can be retracted with a retractor blade attached to a retractor blade holder supported by the retractor support apparatus where the vertical position is adjusted with an articulated joint and the humerus is retracted with a rack and pinion mechanism on the retractor blade holder as disclosed in U.S. Pat. Nos. 6,368,271 and 6,659,944, previously incorporated by reference in their entirety.

The humerus 56 is retracted from the glenoid cavity 52 a selected distance to provide the surgeon access to both the freshly cut surface 57 on the humerus 56 and also to the glenoid cavity 52 within the scapula 51 after the detached humeral ball 54 is separated or dislocated from the glenoid cavity 52.

Alternatively, the humeral ball 54 while attached to the humerus 56 may be first dislocated from the glenoid cavity 52 to gain access to the glenoid cavity 52 for preparation to accept an insert 68. The humerus 56 is laterally retracted, either manually or with a mechanical mechanism as previously disclosed, from the shoulder joint 32 to gain access to both the humeral ball 54 and the glenoid cavity 52 within the scapula 57. The humeral ball 54 is then separated from the humerus 56, typically with a bone saw, thereby creating the surface 57 to which the humeral insert 70 is secured.

The humeral insert 70 that replaces the humeral ball 54 can be shaped to conform to the cut surface 57 of the humerus 56 and cemented into place. The insert 70 may also include a single shaft 72 or a plurality of pegs (not shown) that are inserted to cooperating cavities 72 that are reamed into the humerus 56 and cemented into place. Alternatively, the humeral insert 70 may include mesh-like surface may be positioned onto the humerus 56 or within the cavity 72 reamed into the humerus 56 such that mesh-like surfaces engage the humerus 56 and allow the bone to grow onto the humeral insert 70 to secure the insert 70 to the humerus 56. The humeral insert 70 is preferably constructed of a highly polished stainless steel or titanium.

After the humeral insert 70 is secured to the humerus 56, the scapula 51 having the glenoid cavity 52 is also prepared to accept the prosthetic insert 68 by reaming the scapula 51 such that the glenoid cavity 52 has a similar configuration to an outer surface of the insert 68. One embodiment of the insert 68 that is positioned within the glenoid cavity 52 is constructed from a high density polymer, such as polyethylene, which interacts with the polished humeral insert 70 to reconstruct the shoulder joint 32. The insert 68 preferably has a plurality of pegs 71 that are positioned within cavities reamed into the scapula 51 and intersecting the glenoid cavity 52 where the insert 68 is cemented into place.

Another embodiment of the insert 68 includes two components (not shown), a highly polished metal component that is secured to the scapula 51 having the glenoid cavity 52 and a polymer component that is secured to the metal component and interacts with the humeral insert 70. The metal component may include a shaft (not shown) or a plurality of pegs that engage complimentary indentions that are reamed into the scapula that includes the glenoid cavity where the metal component is cemented to the scapula. Alternatively, the metallic portion insert may include a mesh-like surface that is positioned within the glenoid cavity where the scapula grows onto the insert to secure the metallic portion of the insert to the bone. Although either insert 68 is within the scope of the present invention, the two component insert provides an advantage of replacing only the polymeric portion of the insert without having to perform additional surgery on the scapula 51 in the event the polymeric portion of the insert wears and causes the patient discomfort. With the prosthetic insert secured to the scapula 51 and within the glenoid cavity 52, the humeral insert 70 is reducted into the insert 68 secured within the glenoid cavity 52.

The highly polished humeral insert 70 attached to the humerus 54 engages the high density polymer insert 68 attached to the scapula 51 within the glenoid cavity 52 such that the articulation of the inserts 68, 70 is almost frictionless and resembling the function of a healthy shoulder joint. One skilled in the art will recognize that in a total shoulder-joint replacement surgery all of the cartilage and synovial membrane attached to or positioned between the scapula 51 and the humerus 54 are removed, thereby requiring the nearly frictionless interaction between the inserts 70, 68 attached to the humerus 56 and the scapula 51, respectively, for the reconstructed shoulder joint 32 to function properly.

Although the total shoulder-joint replacement surgery described first prepares the humerus 54 for the humeral insert 70 and then prepares the glenoid cavity 52 within the scapula 51 for the insert 68, it is within the scope of the present invention to first prepare the glenoid cavity 52 within the scapula 51 followed by the humerus 54 for accepting prosthetic inserts 68, 70, respectively. Additionally, it is within the scope of the present invention to replace the damaged end of either the humerus 54 or the glenoid cavity 52 within the scapula 51 with an insert while leaving the undamaged end of the other bone intact.

Once the inserts 70, 68 have been secured to the humerus 54 and the scapula 51 within the glenoid cavity 52, the lateral and medial retractors 36, 44 are removed from the incision 34 and the incision 34 is sutured closed. A drain (not shown) may be positioned within the shoulder joint 32 to remove excess blood and fluids that may accumulate in the shoulder joint 32 caused by the trauma from the total or partial shoulder joint replacement surgery. Once the shoulder joint 32 stops draining, the drain is removed and the incision 34 is completely closed.

Although the present invention has been described with reference to preferred embodiments, workers skilled in the art will recognize that changes may be made in form and detail without departing from the spirit and scope of the invention.

1. A method of performing a shoulder joint surgery involving a scapula having a glenoid cavity, a humerus and a humeral ball on a surgical table, the method comprising:
mounting a retractor support to the surgical table;
positioning the retractor support about the shoulder joint;
incising skin and flesh layers proximate the shoulder joint;
manually retracting the skin and flesh layers proximate the shoulder joint with a retractor; and
securing the retractor in a selected retracting position by attaching the retractor to the retractor support.

2. The method of claim 1 and wherein the retractor support is mounted to a rail of the surgical table.

3. The method of claim 1 and wherein the shoulder joint surgery comprises a shoulder joint replacement surgery wherein an insert is secured within the glenoid cavity and a humeral insert is secured to the humerus.

4. The method of claim 1 and wherein the shoulder joint surgery comprises a partial shoulder joint surgery wherein a humeral insert is secured to the humerus.

5. The method of claim 1 and further comprising:
dislocating the humeral ball from the glenoid cavity;
positioning a humerus retractor about the humerus;
manually retracting the humeral ball from the glenoid cavity; and
securing the humerus retractor to the retractor support.

6. The method of claim 1 and further comprising separating the humeral ball from the humerus with the humeral ball remaining in the glenoid cavity.

7. The method of claim 6 and further comprising removing the humeral ball from the humerus and exposing a freshly cut surface.

8. The method of claim 6 and further comprising:
reaming a cavity into the freshly cut surface of the humerus;
disposing a stem of humeral insert into the cavity in the humerus and wherein a ball is attached to the stem; and
securing the stem within the cavity such that the ball is fixed into a selected position on the humerus.

9. The method of claim 6 and further comprising:
dislocating the humeral ball from the glenoid cavity;
preparing the scapula within the glenoid cavity to accept an insert; and
securing an insert within the glenoid cavity to the scapula.

10. The method of claim 9 and further comprising disposing the ball attached to the humerus within the insert secured within the glenoid cavity.

11. A method of preparing a surgical site for a surgical procedure on a shoulder joint, the method comprising:
mounting a retractor support to the surgical table;
positioning the retractor support about the shoulder joint;
incising skin and flesh layers proximate the shoulder joint;
manually retracting the skin and flesh layers proximate the shoulder joint with a retractor to expose a surgical site; and
attaching the retractor to the retractor support.

12. The method of claim 11 and wherein the retractor support is mounted to a rail of the surgical table.

13. The method of claim 11 and wherein the surgical procedure comprises a shoulder joint replacement surgery wherein an insert is secured within a glenoid cavity of a scapula and a humeral insert is secured to a humerus.

14. The method of claim 11 and wherein the surgical procedure comprises a partial shoulder joint surgery wherein a humeral insert is secured to a humerus.

15. The method of claim 11 and wherein the surgical procedure comprises repairing a damaged muscle, tendon or ligament.

16. The method of claim 11 and wherein the retractor support comprises a generally “J” shaped support.

17. The method of claim 16 and wherein the retractor support further comprises a rod clamped to the “J” shaped support and wherein the rod and the “J” shaped support are positioned on opposite sides of the shoulder.
